(19) United States
(12) Patent Application Publication
(10) Pub. No.: US 2023/0346848 A1
(43) Pub. Date: Nov. 2, 2023

(54) FIBROBLAST AND TLR ACTIVATED FIBROBLAST TREATMENT OF VIRAL INDUCED ACUTE RESPIRATORY DISTRESS SYNDROME

(71) Applicant: FIGENE, LLC, Houston, TX (US)

(72) Inventor: Thomas ICHIM, San Diego, CA (US)

(21) Appl. No.: 17/904,606

(22) PCT Filed: Mar. 2, 2021
(86) PCT No.: PCT/US2021/020449

§ 371 Date: Aug. 19, 2022

Related U.S. Application Data
(60) Provisional application No. 62/986,339, filed on Mar. 6, 2020.

(51) Int. Cl. A61K 35/33; A61K 31/485; A61K 31/55; A61K 31/727; A61K 31/58; A61K 38/48; A61K 38/28; A61K 31/4745; A61K 31/137; A61K 35/28; A61K 31/192; A61P 37/02

(52) U.S. Cl. CPC A61K 35/33; A61K 31/485; A61K 31/55; A61K 31/727; A61K 31/58; A61K 38/4813; A61K 38/28; A61K 31/4745; A61K 31/137; A61K 35/28; A61K 31/192; A61P 37/02

(57) ABSTRACT

Disclosed herein, in certain embodiments, are methods and compositions for treatment of acute respiratory distress syndrome (ARDS) induced by, inter alia, viral agents, comprising administration of fibroblasts, and/or fibroblast derivatives, and/or fibroblast apoptotic bodies. In one embodiment, a patient infected with coronavirus (COVID19) ARDS is administered a concentration of fibroblasts, intravenously, ranging from 10,000 fibroblasts to 300 million fibroblasts, based on patient characteristics and cause of ARDS. In some embodiments, fibroblasts are administered in a non-activated form, whereas in other embodiments, fibroblasts are treated under conditions stimulating enhanced activities beneficial to treatment of ARDS.

This application claims priority to U.S. Provisional Patent Application Ser. No. 62/986,339, filed Mar. 6, 2020, which is incorporated by reference herein in its entirety.

### TECHNICAL FIELD

Embodiments of the disclosure encompass at least the fields of general fields, e.g., cell biology, molecular biology, and medicine.

### BACKGROUND

Acute respiratory distress syndrome (ARDS) is a type of severe acute lung dysfunction affecting all or most of both lungs and can be a severe complication of any number of factors such as pneumonia, sepsis, trauma, or inhaled irritants [1, 2]. Direct and indirect insults to the parenchyma or vasculature of the lungs are typically followed by rapid-onset respiratory failure. ARDS is a serious condition with associated high mortality that afflicts approximately 200,000 people in the United States each year, leading to approximately 75,000 deaths. A number of clinical trials of treatments for ARDS have been conducted and to date none have been proved highly effective; therefore there is a great need for new, more effective treatments.

ARDS is induced by many factors, including bacterial and viral pneumonia, sepsis, inhalation of harmful substances, head, chest or other major injury, burns, blood transfusions, near drowning, aspiration of gastric contents, pancreatitis, intravenous drug use, and abdominal trauma. Furthermore, those with a history of chronic alcoholism are at a higher risk of developing ARDS.

It is known that ARDS is often associated with fluid accumulation in the lungs. When this occurs, the elastic air sacs (alveoli) in the lungs fill with fluid and the function of the alveoli is impaired. The result is that less oxygen reaches the bloodstream, depriving organs of the oxygen required for normal function and viability. In some instances, ARDS occurs in people who are already critically ill or who have significant injuries. Severe shortness of breath, the main symptom of ARDS, usually develops within a few hours to a few days after the precipitating injury or infection.

Many patients who develop ARDS do not survive. The risk of death increases with age and severity of illness. Of the people who do survive ARDS, some recover completely while others experience lasting damage to their lungs.

There are currently no effective pharmacologic therapies for treatment or prevention of ARDS. While inhibition of fibrin formation mitigated injury in some preclinical models of ARDS, anticoagulation therapies in humans do not attenuate ARDS and may even increase mortality. Protective lung ventilator strategies remain the mainstay of available treatment options. Due to the significant morbidity and mortality associated with ARDS and the lack of effective treatment options, new therapeutic agents for the treatment of ARDS and new treatment methods for ARDS are needed.

Some experiments have been conducted using mesenchymal stem cells (MSC) as treatments for ARDS. Unfortunately, the utilization of these cells may not be ideal in conditions of viral induced ARDS because MSC may actually stimulate viral growth and replication, in part through their ability to inhibit the immune system. It is known that these cells promote generation of T regulatory cells [3-31], suppress NK cells [32-36], and act as inhibitors of CD8 cytotoxic T cells [37, 38]. These features could be associated with dampening of antiviral immunity and could serve to reactivate the virus. In fact, detrimental effects of MSC induced immune suppression have already been documented [39, 40], with some studies actually showing that MSC induce viral re-activation [41-43].

The present disclosure addresses the unmet need in the art by providing novel therapeutic agents useful in the treatment of ARDS and methods of treatment for ARDS and conditions related thereto through the administration of such novel therapeutic agents.

### BRIEF SUMMARY

Embodiments of the present disclosure concern treatments and/or prophylaxis of lung dysfunction. Certain embodiments concern treatments and/or prophylaxis of acute lung dysfunction, such as acute respiratory distress syndrome (ARDS). Certain embodiments concern the reprogramming of monocytes in a subject, such as reprogramming monocytes in the lunch of the subject. In some embodiments, a subject encompassed herein has, or is suspected of having, lung dysfunction, such as ARDS. In some embodiments, a subject is administered one or more compositions or one or more treatments encompassed herein. Certain embodiments concern the administration of an effective amount of fibroblasts and/or fibroblast-derived products. In some embodiments, ARDS is caused by one or more factors selected from the group consisting of cytokine storm, immunological cell infiltration, bacterial infection, viral infection, systemic inflammatory response syndrome, systemic inflammation, acute radiation syndrome, sepsis, and a combination thereof.

The disclosure encompasses the use of fibroblasts, and in some cases, toll-like receptor-activated fibroblasts, as a means of reducing ARDS while concurrently producing factors such as interferons in order to stimulate anti-viral immunity.

In some embodiments, fibroblasts of the disclosure are derived from one or more tissues. The tissues may comprise dermal tissue, placental tissue, hair follicle, deciduous tooth, omentum, placenta, Wharton's jelly, bone marrow, adipose tissue, amniotic membrane, amniotic fluid, and/or peripheral blood. In some embodiments, the peripheral blood comprises mobilized peripheral blood to enhance concentration of fibroblasts before isolation of fibroblasts. The mobilized peripheral blood may comprise peripheral blood from an individual that is treated with G-CSF; M-CSF; GM-CSF; Mozibil; flt-3 ligand; or a combination thereof. In some embodiments, the fibroblasts are allogeneic, autologous, xenogeneic, or a combination thereof, with respect to any subject, including a subject administered the fibroblasts and/or fibroblast-derived products.

In some embodiments, fibroblasts are pre-activated with one or more agents capable of enhancing a fibroblast therapeutic activity. The fibroblast therapeutic activity may comprise a mobility towards a chemotactic agent, a production of anti-inflammatory molecules, a production of anti-apoptotic molecules, or a combination thereof. The mobility towards a chemotactic agent may be mediated by enhanced expression of one or more receptors associated with enhanced chemotaxis, including for example CXCR4. The production of anti-inflammatory molecules may comprise the production of molecules selected from the group consisting of IL-4, IL-10, IL-13, IL-20, IL-27, IL-35, PGE-2, indolamine 2,3 deoxygenase, TGF-beta, EGF, and a combination thereof.

In some embodiments, the fibroblasts are modified to express enhanced levels of one or more therapeutic cytokines. The one or more therapeutic cytokines may be cytokines that inhibit apoptosis; cytokines that act as growth factors; cytokines that act as immune modulators and/or anti-inflammatory agents; and a combination thereof. The cytokines that inhibit apoptosis may be, for example, EGF, VEGF, angiopoietin, or a combination thereof. The cytokines that act as growth factors may be, for example, HGF, FGF-1, FGF-2, KGF, CTNF, or a combination thereof. The cytokines that act as immune modulators and/or anti-inflammatory agents may be, for example, IL-4, IL-10, IL-13, IL-20, IL-27, IL-35, PGE-2, indolamine 2,3 deoxygenase, TGF-beta, neuroaminidase, or a combination thereof.

In some embodiments, the fibroblasts are endowed with an ability to suppress a viral infection. The ability to suppress a viral infection may comprise the production of interferon, including interferon alpha, interferon beta, interferon gamma, interferon tau, interferon omega, or a combination thereof. In some embodiments, the ability to suppress viral infection is accomplished by contacting the fibroblasts with an activator of a toll like receptor. The toll like receptor may comprise TLR-1, TLR-2, TLR-3, TLR-4, TLR-5, TLR-6, TLR-7, TLR-8, TLR-9, or a combination thereof. The activator of TLR-1 may comprise Pam3CSK4. The activator of TLR-2 may comprise HKLM. The activator of TLR-3 may comprise Poly:IC. The activator of TLR-4 may comprise LPS, buprenorphine, carbamazepine, fentanyl, levorphanol, methadone, cocaine, morphine, oxcarbazepine, oxycodone, pethidine, glucuronoxylomannan from Cryptococcus, morphine-3-glucuronide, lipoteichoic acid, β-defensin 2, small molecular weight hyaluronic acid, fibronectin EDA, snapin, tenascin C, or a combination thereof. The activator of TLR-5 may comprise flagellin. The activator of TLR-6 may comprise FSL-1. The activator of TLR-7 may comprise imiquimod. The activator of TLR8 may comprise ssRNA40/LyoVec. The activator of TLR-9 may comprise a CpG oligonucleotide, ODN2006, agatolimod, or a combination thereof.

In some embodiments, fibroblast-derived products comprise microvesicles from fibroblasts, exosomes from fibroblasts, apoptotic vesicles from fibroblasts, nucleic acids from fibroblasts, or a combination thereof.

In some embodiments, fibroblasts and/or fibroblast-derived products are administered intravenously, intranasally, intratracheally, or a combination thereof.

In some embodiments, two or more compositions and/or treatments, including any composition and/or treatment encompassed herein, such as fibroblasts and/or fibroblast-derived products, are administered to a subject. The compositions may be administered to the subject sequentially in any order and/or simultaneously. Compositions and/or treatments of the present disclosure that may be provided to a subject in need in addition to fibroblasts and/or fibroblast-derived products comprise at least ventilation, a glucocorticoid, a surfactant, inhaled nitric oxide, an antioxidant, a protease inhibitor, a recombinant human activated protein C, a β2-agonist, lisofylline, a statin, inhaled heparin, a diuretic, a sedative, an analgesic, a muscle relaxant, an antibiotic, inhaled prostacyclin, inhaled synthetic prostacydin analog, ketoconazole, alprostadil, keratinocyte growth factor, beta-agonists, human monoclonal antibody (mAb) against tissue factor VIIa (TS factor 7a), interferon receptor agonists, insulin, perfluorocarbon, budesonide, recombinant human angiotensin-converting enzyme (ACE), recombinant human Clara cell 10 kDa (CC10) protein, tissue plasminogen activator, human mesenchymal stem cells, one or more nutritional therapies, methylprednisolone, dexamethasone, prednisone, prednisolone, betamethasone, triamcinolone, triamcinolone acetonide, beclometasone, albuterol, lisofylline, rosuvastatin, inhaled heparin, inhaled nitric oxide, recombinant human activated protein C, ibuprofen, naproxen, acetaminophen, cisatracurium besylate, procysteine, acetylcysteine, inhaled prostacydin, ketoconazole, alprostadil, keratinocyte growth factor, beta-agonists, human monoclonal antibody (mAb) against tissue factor VIIa (TS factor 7a), insulin, perfluorocarbon, budesonide, a combination of omega-3 fatty acids, antioxidants, gamma-linolenic acids with isocaloric foods, mechanical ventilation, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1 shows stimulation of interferon alpha production from pulmonary epithelial cells by conditioned media from TLR-3-stimulated fibroblasts. The bars for each cell type, from left to right, are control, Poly IC (250 ng), Poly IC (500 ng), and Poly IC (1 μg).

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value. In some embodiments, the term refers to a range of values plus or minus 10 percent, e.g. “about 1.0” encompasses values from 0.9 to 1.1

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete cellular factors that may be useful for any suitable purpose, such as may provide trophic support to other cells. Such factors include, but are not limited to hormones, cytokines, chemokines, extracellular matrix (ECM), proteins, vesicles, antibodies, and granules. In this example, the medium containing the cellular factors is conditioned medium.

As used herein, a “trophic factor” describes a substance that promotes and/or supports survival, growth, proliferation and/or maturation of a cell. Alternatively or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays. One skilled in the art will recognize that the condition of the individual can be monitored throughout the course of therapy and that the effective amount of a compound or composition disclosed herein that is administered can be adjusted accordingly. In some embodiments, the terms refer to a portion of a compound that has a net positive effect on the health and wellbeing of a human or other animal. Therapeutic effects may include an improvement in longevity, quality of life and the like these effects also may also include a reduced susceptibility to developing disease or deteriorating health or wellbeing. The effects may be immediate realized after a single dose and/or treatment or they may be cumulative realized after a series of doses and/or treatments.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

### II. Methods and Compositions for Disease Treatment or Prevention

The disclosure provides treatment of ARDS using fibroblasts and/or fibroblast-derived products. In specific embodiments, the disclosure encompasses treatment of ARDS using fibroblasts, fibroblast apoptotic bodies, and/or fibroblast conditioned media including fibroblast exosomes. In one embodiment the disclosure provides treatment of subjects suffering from viral-associated ARDS, in particular, coronavirus-associated ARDS. Contraction of coronaviruses typically results in respiratory and enteric infections affecting both animals and humans, and was seen as a relatively benign infection. This perception was changed with the severe acute respiratory syndrome (SARS-CoV) outbreak in 2002 and 2003 in China. A decade later, Middle East respiratory syndrome coronavirus (MERS-CoV), another pathogenic coronavirus with a clinical picture reminiscent of SARS, was isolated in patients presenting with pneumonia in the Middle Eastern countries. Just recently, in December 2019, a novel coronavirus (2019-nCoV) emerged in Wuhan, China and has turned into a global health concern. The major cause of death in these situations is cytokine storm associated with ARDS. The disclosure provides at least the use of cellular therapies (and/or products thereof) to reduce, ameliorate, and/or reverse ARDS, including at least one symptom, and including, in some embodiments, through reprogramming of monocytes in the lung, as one example.

In some embodiments, fibroblasts are cultured prior to delivery to a subject in need thereof or prior to production of fibroblast-derived products. In specific embodiments, fibroblasts are cultured with one or more agents capable of enhancing therapeutic properties of the fibroblasts or products derived therefrom. In some embodiments, fibroblasts are cultured with one or more agents capable of enhancing immune modulation properties of the fibroblasts. In some embodiments, fibroblasts are cultured with an agent capable of enhancing monocyte reprogramming properties of the fibroblasts. Fibroblasts may be cultured with one or more agents before providing the fibroblasts to an individual. In some embodiments, fibroblasts are cultured with oxytocin. In some embodiments, fibroblasts are cultured with human chorionic gonadotropin (hCG). In some embodiments, fibroblasts are cultured with agents capable of stimulating immunomodulatory activity, said agents include, for example, one or more activators of toll like receptors. The fibroblasts may be cultured with oxytocin, hCG, and/or one or more activators of toll like receptors.

In some embodiments, fibroblast cells of the present disclosure may be used to secrete one or more angiogenic hormones under conditions useful for reducing fibrosis associated with ARDS. Examples of angiogenic hormones include, but are not limited to, vascular growth factor, endothelial cell growth factor, a combination thereof, and the like. Fibroblasts may be used to induce angiogenesis within a pulmonary tissue in which various progenitor cells are present. Thus, in some embodiments, the disclosure provides a method of promoting neovascularization within a tissue using fibroblasts. The fibroblasts may be introduced to the desired tissue under conditions sufficient for the fibroblasts to produce the angiogenic hormone. The presence of the hormone within the tissue may promote neovascularization within the tissue.

In some embodiments, fibroblasts are provided to an individual via administration to respiratory system, with emphasis on pulmonary systems. The precise mode of administration may be varied depending on factors including disease type, the age of the individual, etc. In some embodiments, fibroblasts are administered to the pulmonary system by arterial, bronchial, inhalable, or other means. In some embodiments, fibroblasts are introduced to the desired site by direct injection. In some embodiments, fibroblasts are administered to the brain of an individual by direct transplantation. In some embodiments, fibroblasts are administered to the pulmonary system of an individual (e.g., the lung) by simple injection.

Aspects of the present disclosure comprise cells useful in therapeutic methods and compositions. Cells disclosed herein include, for example, fibroblasts, stem cells (e.g., hematopoietic stem cells or mesenchymal stem cells), endothelial progenitor cells, and combinations thereof. Cells of a given type (e.g., fibroblasts) may be used alone or in combination with cells of other types for the treatment of ARDS.

Guidance for use of cellular therapies for ARDS may be found in references that describe utilization of cells other than fibroblasts for treatment of this condition. Studies conducted in animal models, and extracorporeal lung models [44, 45] may provide a guidance for various preclinical utilities of fibroblasts in the context of ARDS. Addictionally, numerous types of tissues have been used including mesenchymal stem cells of adipose [46-49], bone marrow [50-69], placental [70], amniotic membrane [71, 72], umbilical cord [73-79], menstrual blood [80], and lung [81, 82], origin, as well as conditioned media [83-90], have demonstrated reduction of pulmonary injury, water leakage, and neutrophil accumulation.

Certain embodiments of the disclosure provide the utilization of fibroblasts, manipulated fibroblasts, fibroblast-derived products, and/or combinations of fibroblasts with other cells, for treatment of ARDS. For example, fibroblasts may be isolated and provided to a subject alone or in combination with one or more stem cells and/or other agents. In one example, fibroblasts are isolated and provided to a subject together with one or more endothelial progenitor cells. In some embodiments, disclosed herein are fibroblasts capable of stimulating tissue regeneration, immune modulation, angiogenesis, and/or neurogenesis. In some embodiments, disclosed herein are fibroblasts capable of stimulating neurogenesis.

In one embodiment, compositions of the present disclosure may be obtained from isolated fibroblast cells or a population thereof capable of proliferating and differentiating into ectoderm, mesoderm, or endoderm. In some embodiments, fibroblasts, including an isolated fibroblast cell population, express at least one of Oct-4, Nanog, Sox-2, KLF4, c-Myc, Rex-1, GDF-3, LIF receptor, CD105, CD117, CD344 or Stella markers. In some embodiments, fibroblasts, including an isolated fibroblast cell population, do not express at least one of MHC class I, MHC class II, CD45, CD13, CD49c, CD66b, CD73, CD105, or CD90 cell surface proteins. In some embodiments, fibroblasts are used as a source of conditioned media. The cells may be cultured alone, or may by cultured in the presence of other cells in order to further upregulate production of growth factors in the conditioned media.

Fibroblasts may be expanded and administered to a subject, or may be cultured in a growth media in order to obtain conditioned media and/or fibroblast-derived products, and these may be administered to a subject. The term Growth Medium generally refers to a medium sufficient for the culturing of fibroblasts. In particular, one particular medium for the culturing of the cells of the disclosure herein comprises Dulbecco's Modified Essential Media (DMEM). In some embodiments, the medium comprises DMEM-low glucose (also DMEM-LG herein) (Invitrogen®, Carlsbad, Calif.). The DMEM-low glucose may be supplemented with 15% (v/v) fetal bovine serum (e.g. defined fetal bovine serum, Hyclone™, Logan Utah), antibiotics/antimycotics (including penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some embodiments, different growth media are used, or different supplementations are provided, and these are normally indicated as supplementations to Growth Medium. Also relating to the present disclosure, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

In some embodiments, fibroblast cells encompassed herein can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. These cell numbers may be produced within 80, 70, or 60 days or less. In one embodiment, fibroblast cells are isolated and expanded, and possess one or more markers selected from the group consisting of CD10, CD13, CD44, CD73, CD90, CD141, PDGFr-alpha, HLA-A, HLA-B, HLA-C, and a combination thereof. In some embodiments, the fibroblast cells do not produce one or more markers selected from the group consisting of CD31, CD34, CD45, CD117, CD141, HLA-DR, HLA-DP, HLA-DQ, and a combination thereof.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be obtained from a source selected from the group consisting of dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and a combination thereof. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGβ3-1) and/or TGβ3-3. Factors from manipulated or stimulated fibroblasts may be present in conditioned media and collected for therapeutic use. The fibroblasts may be manipulated to express any of these or other gene product(s) following transfection or transduction with a vector (viral or non-viral) encoding the gene product(s). Viral vectors include adenoviral, lentiviral, retroviral, and adeno-associated viral vectors.

In some embodiments, fibroblasts are transfected or transduced, or otherwise manipulated to express, one or more angiogenic genes, for example to enhance ability to promote neural repair. An “angiogenic gene” describes a gene encoding for a protein or polypeptide capable of stimulating or enhancing angiogenesis in a culture system, tissue, or organism. Examples of angiogenic genes which may be useful in embodiments encompassed herein include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shingoingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of treatment or prevention of ARDS.

Under appropriate conditions, fibroblasts may be capable of producing interleukin-1 (IL-1) and/or other inflammatory cytokines. In some embodiments, fibroblasts of the present disclosure are modified (e.g., by gene editing or other methods capable of preventing or reducing expression of a protein) to prevent or reduce expression of IL-1 or other inflammatory cytokines. In some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual (e.g., into pulmonary cells of an individual). In some embodiments, fibroblasts are subjected to differentiation into a different cell type prior to introduction into the individual (e.g., into the lung system).

As disclosed herein, fibroblasts may secrete one or more factors prior to and/or following introduction into an individual. Such factors include, but are not limited to, growth factors, trophic factors and/or cytokines. The fibroblasts may or may not be manipulated to secrete such factors. In some instances, the secreted factors can have a therapeutic effect in the individual. In some embodiments, a secreted factor activates a particular cell. In some embodiments, the secreted factor activates neighboring and/or distal endogenous cells. In some embodiments, the secreted factor stimulates cell proliferation and/or cell differentiation. In some embodiments, fibroblasts secrete one or more cytokines and/or one or more growth factors, such as those selected from human growth factor, fibroblast growth factor, nerve growth factor, insulin-like growth factors, hemopoietic stem cell growth factors, a member of the fibroblast growth factor family, a member of the platelet-derived growth factor family, a vascular or endothelial cell growth factor, and a member of the TGFβ family, for example.

In some embodiments, the administration route of the fibroblasts is relevant to achieve therapeutic efficacy. For instance, in the treatment of ARDS, a pharmaceutical composition comprising fibroblasts and/or fibroblast-derived products, such as extracellular vesicles derived from the fibroblasts and/or apoptotic bodies of the fibroblasts, may be administered via peripheral intravenous injection, central venous injection into the right atrium, injection into the right ventricle of the heart, and/or injection into the pulmonary trunk/artery. In some embodiments, the pharmaceutical compositions as per the present disclosure display therapeutic efficacy in the patient group that (1) suffer from ARDS, Infant respiratory distress syndrome (IRDS), Pulmonary hypertension (PH), or any other pulmonary disease or disorder within the scope of the present disclosure, and (2) are eligible and/or are undergoing extra-corporal membranous oxygenation (ECMO) treatment. The pharmaceutical compositions in accordance with the present disclosure can thus be administered both to patients that have already been placed on ECMO support, and to patients that are eligible but have not yet commenced ECMO treatment, in some cases.

The present disclosure, in a further aspect, thus relates to the use of the pharmaceutical compositions according to the present disclosure for use in medicine, and specifically in the treatment of diseases and disorders such as ARDS, IRDS, PH, congenital heart diseases, and acute organ failure (optionally in connection with ARDS and/or IRDS), for instance heart failure, kidney failure, and/or liver failure.

ARDS is an important cause of acute respiratory failure and is often associated with multiple organ failure. Several clinical disorders can precipitate ARDS, for instance viral and/or bacterial pneumonia, aspiration of gastric contents, sepsis, surgery, and major trauma. The clinical criteria for ARDS may include the following: acute onset: 20-50% of acute lung injury patients will develop ARDS within 7 days; capillary wedge pressure (PCWP) 518 mmHg or no evidence of cardiac failure; chest X-ray shows bilateral infiltrates; refractory hypoxaemia: ARDS is present when PaO₂:FiO₂<200. Physiologically, ARDS may be characterized by increased permeability pulmonary edema, severe arterial hypoxemia, and impaired carbon dioxide excretion and is the result of an on-going inflammatory response. [0105] Clinically, up-regulation of inflammatory cytokines frequently persists in the patients.

Infant respiratory distress syndrome (IRDS) of the newborn is the most common cause of respiratory distress in premature infants, correlating with structural and functional lung immaturity. The pathophysiology is characterized by an ongoing inflammatory response giving immature type II alveolar cells that produce less surfactant, causing an increase in alveolar surface tension and a decrease in compliance. The resultant atelectasis causes pulmonary vascular constriction, hypoperfusion, and lung tissue ischemia. Hyaline membranes form through the combination of sloughed epithelium, protein, and edema. Persistent respiratory distress syndrome leads to bronchopulmonary dysplasia, characterized by typical chest radiography findings and chronic oxygen dependence.

Pulmonary hypertension (PH) is an increase in blood pressure in the pulmonary artery, pulmonary vein, and/or the pulmonary capillaries and it can be a severe disease with a markedly decreased exercise tolerance and heart failure. Evidence is accumulating to suggest that inflammation plays a significant role in the pathogenesis of PH. Endothelial cells play an important role in inflammation and immune reactions, and inflammatory cytokines cause endothelial dysfunction. Endothelial dysfunction is a hallmark of PH, consisting in reduced availability of vasodilators and antiproliferative factors and increased production of vasoconstrictors and vascular proliferative factors. Up-regulation of inflammatory cytokines and perivascular inflammatory cell infiltration have been detected in the lungs of patients with PH. Persistent PH of the newborn occurs when pulmonary vascular resistance fails to decrease soon after birth as with normal transition. The etiology may be idiopathic or secondary to meconium aspiration syndrome, pneumonia or sepsis, respiratory distress syndrome, or transient tachypnea of the newborn. The increased pulmonary hypertension gives rise to an ongoing inflammatory response in the lung.

In one embodiment of the disclosure, stimulation of fibroblasts is performed by treatment with one or more activators of a toll like receptor prior to administration of fibroblasts. Numerous toll like receptors may be activated for use in the current disclosure. In certain embodiments, toll like receptors which recognize RNA, similar to ones which are activated by viruses, are utilized. In certain embodiments, toll-like receptor 3 activators are administered to fibroblasts. Specific toll like receptors including Poly-IC.

The outbreak of novel coronavirus (2019-nCoV)—now known as Coronavirus Disease (COVID-19), has been declared by the WHO as a global health emergency. Initial infection occurs in the respiratory tract, in which the virus incubates until inducing carrier status, and/or pathological infection in the host. COVID-19 mortality is associated with acute respiratory distress syndrome (ARDS) and multiple organ failure [91, 92]. What is needed are treatments that can inhibit the infection at the site of entry and suppress viral propagation so as to prevent progression to advanced state.

Since it is known that patients with stronger lung-resident immune responses are more resilient to COVID-19 as compared to ones with weaker immunity, means of boosting localized immunity are urgently needed. Methods of the disclosure include use of fibroblasts and/or fibroblast-derived products for boosting localized immunity in an individual in need thereof, such as one with a weakened immune system.

When cells are infected with viruses, they produce a family of chemicals called “interferons”, which “interfere” with ability of the virus to infect surrounding cells. It has been known since the 1957 that interferon production is a unique biological response to viral infection, which induces production against a broad variety of viral pathogens [93, 94]. Protection against viruses occurs through mechanisms of directly blocking the virus from replicating inside cells [95, 96], as well as stimulation of local immunity including antigen presenting cells [97-103], T cells [104-108], and natural killer (NK) cells [102, 109-116].

Interferon is viewed as the “First Responder” against global viral outbreaks [117]. Clinical trials and case reports support the efficacy of interferon therapy in deadly viral diseases including Ebola [118-120], Marburg [121], and Coronaviruses [122-126].

The dosages of interferons currently used for viral infections are extremely high, due in part, for need to administer systemically. To date, direct intra-pulmonary delivery has not been utilized. The current doses of interferon utilized appear to possess other side effects. Additionally, current interferons utilized are recombinant and do not represent the naturally occurring “symphony of cytokines” that occurs during a physiological anti-viral response.

In some embodiments, fibroblasts treated with TLR agonists, particularly TLR3 agonists, are utilized, including as a combination therapy with NK cells. In one embodiment, NK cells may be generated in vitro, as described below, and admixed with TLR-3 activated fibroblasts in vitro enhance NK proliferation and cytotoxic activity in vitro. In other embodiments, activated fibroblasts are administered in vivo together with NK cells. The cord blood may be utilized as a source of cytotoxic T cells and/or NK cells. In certain embodiments, NK cells, pharmaceutical compositions comprising the NK cells, and/or any cell therapy of the present disclosure comprise a solution for suspending or culturing living cells, including for example, a saline, a phosphate buffered saline (PBS), a medium, a serum or the like in general. The solution may comprise a carrier pharmaceutically acceptable as a pharmaceutical or a quasi-pharmaceutical in some cases. In certain embodiments, NK cells, pharmaceutical compositions comprising the NK cells, and/or any cell therapy of the present disclosure can be applied to treatment and/or prevention of various diseases having sensitivity to NK cells. Examples of such diseases include, but are not limited to, cancers and tumors such as an oral cancer, a gallbladder cancer, a cholangiocarcinoma, a lung cancer, a liver cancer, a colorectal cancer, a kidney cancer, a bladder cancer and leukemia, and infectious diseases caused by viruses, bacteria and the like. The pharmaceutical composition containing the NK cells of the present disclosure may contain, in addition to the NK cells, an NK cell precursor, T cells, NKT cells, hematopoietic precursor cells and other cells in some cases. The cell therapy of the present disclosure may be practiced singly or in combination with surgical treatment, chemotherapy, radiation therapy or the like in some cases. In the cell therapy of the present disclosure, the NK cells expanded by the method of the present disclosure may be transplanted into a patient together with T cells and NKT cells in some cases.

In some embodiments, the cell therapy of the present disclosure, the cells may be transplanted by, for example, intravenous, intraarterial, subcutaneous or intraperitoneal administration in some cases. In a method for preparing cells of the present disclosure, in the method for preparing the pharmaceutical composition of the present disclosure and in the cell therapy of the present disclosure, any of media such as, but not limited to, a KBM501 medium (Kohjin Bio Co., Ltd.), a CellGro SCGM medium (registered trademark, Cellgenix, Iwai Chemicals Company), a STEMLINE II (Sigma-Aldrich Co. LLC.), an X-VIVO15 medium (Lonza, Takara Bio Inc.), IMDM, MEM, DMEM and RPMI-1640 may be singly used as or blended in an appropriate ratio to be used as a medium for culturing cells in some cases. Besides, the media for culturing cells may be used with supplementation of at least one additional component selected from the group consisting of a serum, a serum albumin, an appropriate protein, a cytokine, an antibody, a compound and another component.

The medium may be supplemented with an autologous serum of a subject, a human AB-type serum available from BioWhittaker Inc. or the like, or a donated human serum albumin available from Japanese Red Cross Society in some cases. The autologous serum and the human AB-type serum may be supplemented in a concentration of 1 to 10%, and the donated human serum albumin may be supplemented in a concentration of 1 to 10%. The subject may be a healthy person, or a patient having any of various diseases sensitive to NK cells. The medium may be supplemented with an appropriate protein, a cytokine, an antibody, a compound or another component as long as the effect of expanding NK cells is not impaired. The cytokine may be interleukin 2 (IL-2), interleukin 3 (IL-3), interleukin 7 (IL-7), interleukin 12 (IL-12), interleukin 15 (IL-15), interleukin 21 (IL-21), stem cell factor (SCF), thrombopoietin (TPO) and/or FMS-like tyrosine kinase 3 ligand (Flt3L) in some cases. The IL-2, IL-3, IL-7, IL-12, IL-15, IL-21, SCF, TPO and Flt3L comprise a human amino acid sequence, and are produced by a recombinant DNA technology from the safety viewpoint. The IL-15 may be used in a concentration of 0.1 to 100 ng/mL, such as in a concentration of 20 to 30 ng/mL, and in some cases in a concentration of 25 ng/mL. The SCF may be used in a concentration of 2 to 100 ng/mL, including in a concentration of 20 to 30 ng/mL, and such as in a concentration of 25 ng/mL. The IL-7 may be used in a concentration of 0.5 to 100 ng/mL, including in a concentration of 20 to 30 ng/mL, and such as in a concentration of 25 ng/mL. The Flt3L may be used in a concentration of 1 to 100 ng/mL, including in a concentration of 20 to 30 ng/mL, and such as in a concentration of 25 ng/mL. The TPO may be used in a concentration of 1 to 100 ng/mL, including in a concentration of 20 to 30 ng/mL, and such as in a concentration of 25 ng/mL. Herein, the concentration of the IL-2 may be shown in Japanese Reference Unit (JRU) and International Unit (IU). Since 1 IU corresponds to approximately 0.622 JRU, 1750 JRU/mL corresponds to approximately 2813 IU/mL. The IL-2 may have a human amino acid sequence and may be produced by a recombinant DNA technology from the safety viewpoint. The IL-2 may be used in a concentration of 100 to 2900 IU/mL, such as in a concentration of 100 to 2813 IU/mL, or such as 2813 IU/mL. In some embodiments, the preparation method of the present disclosure and the cell therapy of the present disclosure, in the step of expanding hematopoietic precursor cells, the cells are cultured in a medium supplemented with IL-15, SCF, IL-7 and Flt3L. The medium may be supplemented further with TPO in some cases. The medium may be replaced at any time after starting the cultivation as long as a desired number of cells can be obtained, and may be replaced every 3 to 5 days, for example. In the expansion of hematopoietic precursor cells, the cell growth rate is abruptly lowered in about 5 weeks. Therefore, the expansion of the hematopoietic precursor cells is conducted for about 5 weeks, namely, for 32, 33, 34, 35, 36, 37 or 38 days, after starting the cultivation. Thereafter, from the expanded hematopoietic precursor cells, NK cells are differentiation induced. In the step of differentially inducing NK cells, the cells are cultured in a medium supplemented with IL-2. The differentiation induction of NK cells is conducted for about 1 week, namely, for 5, 6, 7, 8 or 9 days. Here, cultivation conducted for n days under a given culturing condition refers to that the cultivation is conducted from a cultivation start date to n days after under the culturing condition, and means that transition to a next culturing condition or cell collection is performed n days after starting the cultivation. In the present disclosure, the hematopoietic precursor cells may be frozen during the expansion or after completing the expansion, and thawed in accordance with a time of transplantation into a patient to be used for the transplantation into the patient in some cases. The cells may be frozen and thawed by any of methods known to those skilled in the art. For freezing the cells, any of commercially available cryopreservation solutions is used in some cases.

In the expansion method of the present disclosure, the culture vessel includes, but is not limited to, commercially available dishes, flasks, plates and multi-well plates. The culturing condition is not especially limited as long as the effect of expanding NK cells is not impaired, but a culturing condition of 37° C., 5% CO₂ and a saturated water vapor atmosphere is generally employed. Since the purpose of the present disclosure is to prepare a large amount of NK cells, it is advantageous that the time period of culturing the cells in the medium is longer because a larger amount of NK cells can be thus obtained. The culture period is not especially limited as long as the NK cells can be expanded to a desired number of cells.

In certain embodiments, the method and the production of compositions of the present disclosure are practiced under conditions complying with good manufacturing practices (GMP) for pharmaceuticals and quasi-pharmaceuticals. The cytotoxic activity of cells encompassed herein are evaluated by a method known to those skilled in the art. In some embodiments, the cytotoxic activity of cells is quantitatively determined by incubating the cells (such as effector cells) and target cells labeled with a radioactive substance, a fluorescent dye or the like, and then measuring a radiation dose or a fluorescence intensity. The target cells may be K562 cells, acute myelogenous leukemia cells, or chronic myelogenous leukemia cells in some cases, but are not limited to these. The properties of the cells encompassed herein may be checked by employing RT-PCR, solid phase hybridization, ELISA, Western blotting, immune precipitation, immunonephelometry, FACS, flow cytometry or the like in some cases. In the present disclosure, the collection and cryopreservation of an umbilical cord blood and/or adult blood cell tissue, the preparation of an autologous serum, the preparation of an umbilical cord blood and/or adult blood cell tissue, and mononuclear cells differentiation induced from pluripotent stem cells such as induced pluripotent stem cells, embryonic stem cells or adult stem cells, the preparation of hematopoietic precursor cells from the mononuclear cells, the measurement of the number of cells before and after the cultivation of the hematopoietic precursor cells, the measurement of a constituent ratio among NK cells, T cells and other cell types in the hematopoietic precursor cells before and after the cultivation, the calculation of the expansion factor of the NK cells, and the statistical analysis of a measurement error or significance may be practiced by any methods known to those skilled in the art.

Certain aspects of the present disclosure provide cell therapy. In certain embodiments, the cell therapy of the present disclosure includes a step of expanding hematopoietic precursor cells under a single culturing condition; and optionally a step of differentially inducing the cells obtained in the expanding step into a desired cell type, such as NK cells. In the cell therapy, a medium used in the step of expanding hematopoietic precursor cells under a single culturing condition may be supplemented with IL-15, SCF, IL-7 and Flt3L in some cases. In the cell therapy, the medium used in the step of expanding hematopoietic precursor cells under a single culturing condition may be supplemented further with TPO in some cases. In the cell therapy, the step of differentially inducing the cells, such as NK cells, may include culturing the expanded hematopoietic precursor cells under a culturing condition containing IL-2 in some cases. In the cell therapy, the medium used in each of the steps may be supplemented with a human AB-type serum and/or a human serum albumin. In some embodiments, the cell therapy, the hematopoietic precursor cells are at least one of hematopoietic precursor cells selected from the group consisting of hematopoietic precursor cells contained in an umbilical cord blood and/or an adult blood cell tissue, hematopoietic precursor cells differentiation induced from induced pluripotent stem cells, embryonic stem cells and/or adult stem cells, and hematopoietic precursor cells directly converted from differentiated cells. In certain embodiments, the cell therapy, the step of transplanting the NK cells into a patient may be a step of transplanting the NK cells together with other cells such as T cells or NKT cells in some cases. The cell therapy of the present disclosure may be employed for treating and/or preventing an infectious disease and/or a cancer.

Production of NK cells by the present method comprises expanding a population of hematopoietic cells. During cell expansion, a plurality of hematopoietic cells within the hematopoietic cell population differentiate into NK cells. In one embodiment, provided herein is a method of producing a population of activated natural killer (NK) cells, comprising: (a) seeding a population of hematopoietic stem or progenitor cells in a first medium comprising interleukin-15 (IL-15) and, optionally, one or more of stem cell factor (SCF) and interleukin-7 (IL-7), wherein said IL-15 and optional SCF and IL-7 are not comprised within an undefined component of said medium, such that the population expands, and a plurality of hematopoietic stem or progenitor cells within said population of hematopoietic stem or progenitor cells differentiate into NK cells during said expanding; and (b) expanding the cells from step (a) in a second medium comprising interleukin-2 (IL-2), to produce a population of activated NK cells. In another embodiment, NK cells provided herein are produced by a two-step process of expansion/differentiation and maturation of NK cells. The first and second steps comprise culturing the cells in media with a unique combination of cellular factors. In certain embodiments, the process involves (a) culturing and expanding a population of hematopoietic cells in a first medium, wherein a plurality of hematopoietic stem or progenitor cells within the hematopoietic cell population differentiate into NK cells; and (b) expanding the NK cells from step (a) in a second medium, wherein the NK cells are further expanded and differentiated, and wherein the NK cells are maturated (e.g., activated or otherwise possessing cytotoxic activity). In certain embodiments, the method includes no intermediary steps between step (a) and (b), no additional culturing steps prior to step (a), and/or no additional steps (e.g., maturation step) after step (b). In certain embodiments, the methods provided herein comprises a first step of culturing and expanding a population of hematopoietic cells in a first medium, wherein a plurality of hematopoietic stem or progenitor cells within the hematopoietic cell population differentiate into NK cells. Without wishing to be bound by any parameter, mechanism or theory, culture of the hematopoietic cells as provided herein results in continuous expansion of the hematopoietic cells and differentiation of NK cells from said cells. In certain embodiments, hematopoietic cells, e.g., stem cells or progenitor cells, used in the methods provided herein are expanded and differentiated in the first step using a feeder layer. In other embodiments, hematopoietic cells, e.g., stem cells or progenitor cells, are expanded and differentiated in the first step without the use of a feeder layer. Feeder cell-independent expansion and differentiation of hematopoietic cells can take place in any container compatible with cell culture and expansion, e.g., flask, tube, beaker, dish, multiwell plate, bag or the like. In a specific embodiment, feeder cell-independent expansion of hematopoietic cells takes place in a bag, e.g., a flexible, gas-permeable fluorocarbon culture bag (for example, from American Fluoroseal). In a specific embodiment, the container in which the hematopoietic cells are expanded is suitable for shipping, e.g., to a site such as a hospital or military zone wherein the expanded NK cells are further expanded and differentiated.

### III. Fibroblasts and Cultured Cells

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells or other cells encompassed herein. In some embodiments, disclosed herein are fibroblasts capable of preventing, reducing, and/or treating lung dysfunction, such as ARDS. In some embodiments, fibroblasts of the present disclosure are adherent to plastic. In some embodiments, the fibroblasts express CD73, CD90, and/or CD105. In some embodiments, the fibroblasts are CD14, CD34, CD45, and/or HLA-DR negative. In some embodiments, the fibroblasts possess the ability to differentiate to osteogenic, chondrogenic, and adipogenic lineage cells.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media.

In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, .alpha.-1-Antriptrypsin, Ceruloplasmin, AFP, PEPCK 1, BDNF, NT-4/5, TrkA, BMP2, BMP4, FGF2, FGF4, PDGF, PGF, TGF.alpha., TGFβ, and/or VEGF.

Fibroblasts may be expanded and utilized by administration themselves, or may be cultured in a growth media in order to obtain conditioned media. The term Growth Medium generally refers to a medium sufficient for the culturing of fibroblasts. In particular, one presently medium for the culturing of the cells of the disclosure herein comprises Dulbecco's Modified Essential Media (DMEM). One example is DMEM-low glucose (also DMEM-LG herein) (Invitrogen®, Carlsbad, Calif.). The DMEM-low glucose is supplemented with 15% (v/v) fetal bovine serum (e.g. defined fetal bovine serum, Hyclone™, Logan Utah), antibiotics/antimycotics (such as penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present disclosure, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷, or more cells when seeded at from about 10³ to about 10⁶ cells/cm² in culture. In at least some cases, these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be fibroblasts obtained from various sources including, for example, dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and fibroblasts obtained from a plastic surgery-related by-product. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGβ3-1) and/or TGβ3-3. Factors from manipulated or stimulated fibroblasts may be present in conditioned media and collected for therapeutic use.

In some embodiments, fibroblasts are transfected with one or more angiogenic genes for any purpose, including to enhance ability to promote neural repair. An “angiogenic gene” describes a gene encoding for a protein or polypeptide capable of stimulating or enhancing angiogenesis in a culture system, tissue, or organism. Examples of angiogenic genes that may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and/or EG-VEGF. Fibroblasts transfected with one or more angiogenic factors may be used in the disclosed methods of disease treatment or prevention.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

As disclosed herein, fibroblasts may secret one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts of the present disclosure are cultured with an inhibitor of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/L, 4.5 g/L, 4 g/L, 3 g/L, 2 g/L, or 1 g/L. In some embodiments, such conditions comprise glucose of about 1 g/L.

Aspects of the present disclosure comprise generating conditioned media from fibroblasts. Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500×g). Conditioned media may be filtered through a membrane. The membrane may be a >1000 kDa membrane. Conditioned media may be subject to liquid chromatography such as HPLC. Conditioned media may be separated by size exclusion.

In some embodiments, the present disclosure utilizes exosomes derived from fibroblasts as a therapeutic modality, including embodiments concerning fibroblast-derived products. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/mL and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, preventing, reducing, and/or treating lung dysfunction, such as ARDS. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

### IV. Administration of Therapeutic Compositions

The therapy provided herein may comprise administration of a therapeutic agents (e.g., fibroblasts, exosomes from fibroblasts, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts and/or fibroblast-derived products) of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 mg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

### V. Kits of the Disclosure

Any of the cellular and/or non-cellular compositions described herein or similar thereto may be comprised in a kit. In a non-limiting example, one or more reagents for use in methods for preparing and/or using fibroblasts, fibroblast-derived products, or derivatives thereof (e.g., exosomes derived from fibroblasts) may be comprised in a kit. Such reagents may include cells, one or more growth factors, vector(s) one or more costimulatory factors, media, enzymes, buffers, nucleotides, salts, primers, compounds, and so forth. Any composition encompassed herein may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and in some embodiments, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Stimulation of Interferon Alpha Production from Pulmonary Epithelial Cells by Conditioned Media from TLR-3-Stimulated Fibroblasts

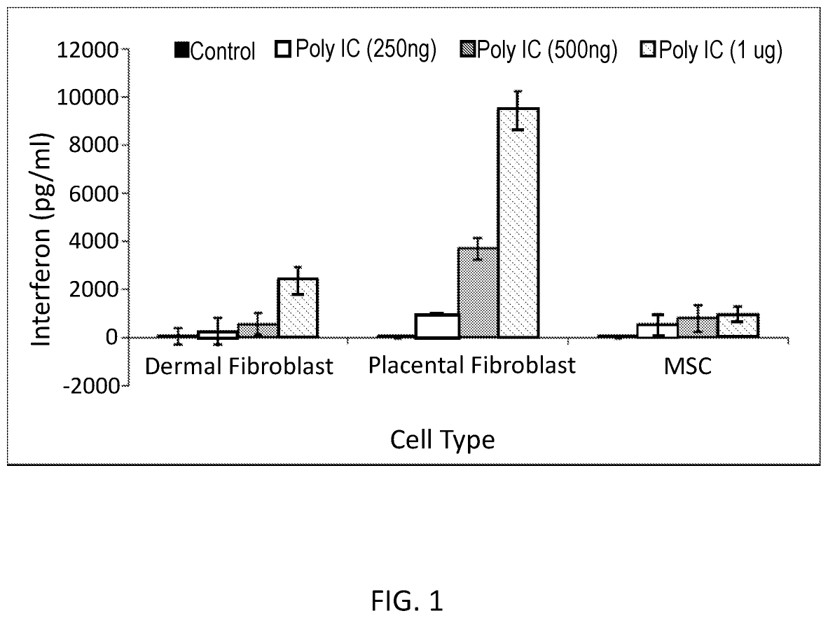

Dermal fibroblasts, placental fibroblasts, and mesenchymal stem cells were treated with different concentrations of Poly IC (0 ng, 250 ng, 500 ng, and 1 μg). The conditioned media from such cells were used to stimulate pulmonary epithelial cells. Amount of interferon from each group of pulmonary epithelial cells were measured. As shown in FIG. 1, cells treated with conditioned media from placental and dermal fibroblasts showed high levels of interferon.

### REFERENCES

All patents and publications mentioned in the specification are indicative of the level of those skilled in the art to which the disclosure pertains. All patents and publications are herein incorporated by reference herein to the same extent as if each individual publication was specifically and individually indicated to be incorporated by reference.
1. Madershahian, N., et al., Application of ECMO in multitrauma patients with ARDS as rescue therapy. J Card Surg, 2007. 22(3): p. 180-4.
2. Villar, J., H. Zhang, and A. S. Slutsky, Lung Repair and Regeneration in ARDS: Role of PECAM1 and Wnt Signaling. Chest, 2019. 155(3): p. 587-594.
3. Court, A. C., et al., Mitochondrial transfer from MSCs to T cells induces Treg differentiation and restricts inflammatory response. EMBO Rep, 2020. 21(2): p. e48052.
4. Liu, Y., et al., Human umbilical cord mesenchymal stem cells confer potent immunosuppressive effects in Sjogren's syndrome by inducing regulatory T cells. Mod Rheumatol, 2020: p. 1-11.
5. Fakhimi, M., et al., Helios, CD73 and CD39 Induction in Regulatory T Cells Exposed to Adipose Derived Mesenchymal Stem Cells. Cell J, 2020. 22(2): p. 236-244.
6. Khosravi, M., et al., Induction of CD4(+)CD25(+)FOXP3(+) regulatory T cells by mesenchymal stem cells is associated with modulation of ubiquitination factors and TSDR demethylation. Stem Cell Res Ther, 2018. 9(1): p. 273.
7. Roux, C., et al., Immunosuppressive Mesenchymal Stromal Cells Derived from Human-Induced Pluripotent Stem Cells Induce Human Regulatory T Cells In vitro and In vivo. Front Immunol, 2017. 8: p. 1991.
8. Miyagawa, I., et al., Regulatory Mechanism of The Induction of Regulatory T Cells through Growth Factors Released by Human Mesenchymal Stem Cells. Crit Rev Immunol, 2018. 38(6): p. 471-478.
9. Khosravi, M., et al., Induction of CD4(+)CD25(+)Foxp3(+) regulatory T cells by mesenchymal stem cells is associated with RUNX complex factors. Immunol Res, 2018. 66(1): p. 207-218.
10. Khosravi, M., et al., Mesenchymal stem cells can induce regulatory T cells via modulating miR-126a but not miR-10a. Gene, 2017. 627: p. 327-336.
11. Lee, H. J., et al., ICOSL expression in human bone marrow-derived mesenchymal stem cells promotes induction of regulatory T cells. Sci Rep, 2017. 7: p. 44486.
12. Chen, C., et al., Mesenchymal stem cells upregulate Treg cells via sHLA-G in SLE patients. Int Immunopharmacol, 2017. 44: p. 234-241.
13. Lim, J. Y., et al., Enhanced immunoregulation of mesenchymal stem cells by IL-10-producing type 1 regulatory T cells in collagen-induced arthritis. Sci Rep, 2016. 6: p. 26851.
14. Lee, E. S., et al., Adoptive Transfer of Treg Cells Combined with Mesenchymal Stem Cells Facilitates Repopulation of Endogenous Treg Cells in a Murine Acute GVHD Model. PLoS One, 2015. 10(9): p. e0138846.
15. Cahill, E. F., et al., Jagged-1 is required for the expansion of CD4+CD25+FoxP3+ regulatory T cells and tolerogenic dendritic cells by murine mesenchymal stromal cells. Stem Cell Res Ther, 2015. 6: p. 19.
16. Wang, Z. X., et al., Mesenchymal stem cells alleviate atherosclerosis by elevating number and function of CD4(+)CD25 (+)FOXP3 (+) regulatory T-cells and inhibiting macrophage foam cell formation. Mol Cell Biochem, 2015. 400(1-2): p. 163-72.
17. Takahashi, T., et al., Multipotent mesenchymal stromal cells synergize with costimulation blockade in the inhibition of immune responses and the induction of Foxp3+ regulatory T cells. Stem Cells Transl Med, 2014. 3(12): p. 1484-94.
18. Frazier, T. P., et al., Human adipose-derived stromal/stem cells induce functional CD4+CD25+FoxP3+CD127-regulatory T cells under low oxygen culture conditions. Stem Cells Dev, 2014. 23(9): p. 968-77.
19. Li, J. G., et al., Human mesenchymal stem cells elevate CD4+CD25+CD127low/− regulatory T cells of asthmatic patients via heme oxygenase-1. Iran J Allergy Asthma Immunol, 2013. 12(3): p. 228-35.
20. Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65.
21. Melief, S. M., et al., Multipotent stromal cells induce human regulatory T cells through a novel pathway involving skewing of monocytes toward anti-inflammatory macrophages. Stem Cells, 2013. 31(9): p. 1980-91.
22. Erkers, T., et al., Decidual stromal cells promote regulatory T cells and suppress alloreactivity in a cell contact-dependent manner. Stem Cells Dev, 2013. 22(19): p. 2596-605.
23. Engela, A. U., et al., Human adipose-tissue derived mesenchymal stem cells induce functional de-novo regulatory T cells with methylated FOXP3 gene DNA. Clin Exp Immunol, 2013. 173(2): p. 343-54.
24. Tasso, R., et al., Mesenchymal stem cells induce functionally active T-regulatory lymphocytes in a paracrine fashion and ameliorate experimental autoimmune uveitis. Invest Ophthalmol Vis Sci, 2012. 53(2): p. 786-93.
25. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
26. Sun, J., et al., Intrapulmonary delivery of human umbilical cord mesenchymal stem cells attenuates acute lung injury by expanding CD4+CD25+ Forkhead Boxp3 (FOXP3)+ regulatory T cells and balancing anti-and pro-inflammatory factors. Cell Physiol Biochem, 2011. 27(5): p. 587-96.
27. Madec, A. M., et al., Mesenchymal stem cells protect NOD mice from diabetes by inducing regulatory T cells. Diabetologia, 2009. 52(7): p. 1391-9.
28. English, K., et al., Cell contact, prostaglandin E(2) and transforming growth factor beta 1 play non-redundant roles in human mesenchymal stem cell induction of CD4+CD25(High) forkhead box P3+ regulatory T cells. Clin Exp Immunol, 2009. 156(1): p. 149-60.
29. Gonzalez-Rey, E., et al., Human adipose-derived mesenchymal stem cells reduce inflammatory and T cell responses and induce regulatory T cells in vitro in rheumatoid arthritis. Ann Rheum Dis, 2010. 69(1): p. 241-8.
30. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
31. Prevosto, C., et al., Generation of CD4+ or CD8+ regulatory T cells upon mesenchymal stem cell-lymphocyte interaction. Haematologica, 2007. 92(7): p. 881-8.
32. Ribeiro, A., et al., Mesenchymal stem cells from umbilical cord matrix, adipose tissue and bone marrow exhibit different capability to suppress peripheral blood B, natural killer and T cells. Stem Cell Res Ther, 2013. 4(5): p. 125.
33. Selmani, Z., et al., Human leukocyte antigen-G5 secretion by human mesenchymal stem cells is required to suppress T lymphocyte and natural killer function and to induce CD4+CD25highFOXP3+ regulatory T cells. Stem Cells, 2008. 26(1): p. 212-22.
34. Shokri, M. R., et al., Human menstrual blood-derived stromal/stem cells modulate functional features of natural killer cells. Sci Rep, 2019. 9(1): p. 10007.
35. Hu, C. D., et al., Differential Immunomodulatory Effects of Human Bone Marrow-Derived Mesenchymal Stromal Cells on Natural Killer Cells. Stem Cells Dev, 2019. 28(14): p. 933-943.
36. Ishida, N., et al., Cotransplantation of preactivated mesenchymal stem cells improves intraportal engraftment of islets by inhibiting liver natural killer cells in mice. Am J Transplant, 2019. 19(10): p. 2732-2745.
37. Pianta, S., et al., Amniotic membrane mesenchymal cells-derived factors skew T cell polarization toward Treg and downregulate Th1 and Th17 cells subsets. Stem Cell Rev Rep, 2015. 11(3): p. 394-407.
38. Hong, J., et al., Indoleamine 2,3-dioxygenase mediates inhibition of virus-specific CD8(+) T cell proliferation by human mesenchymal stromal cells. Cytotherapy, 2016. 18(5): p. 621-9.
39. Lin, R., et al., Bone marrow-derived mesenchymal stem cells favor the immunosuppressive T cells skewing in a Helicobacter pylori model of gastric cancer. Stem Cells Dev, 2013. 22(21): p. 2836-48.
40. Chen, J., et al., Human mesenchymal stem cells promote tumor growth via MAPK pathway and metastasis by epithelial mesenchymal transition and integrin alpha5 in hepatocellular carcinoma. Cell Death Dis, 2019. 10(6): p. 425.
41. Qu, M., et al., Bone Marrow-Derived Mesenchymal Stem Cells Attenuate Immune-Mediated Liver Injury and Compromise Virus Control During Acute Hepatitis B Virus Infection in Mice. Stem Cells Dev, 2017. 26(11): p. 818-827.
42. Malcherek, G., et al., Mesenchymal stromal cells inhibit proliferation of virus-specific CD8(+) T cells. Leukemia, 2014. 28(12): p. 2388-94.
43. Calkoen, F. G., et al., Mesenchymal stromal cell therapy is associated with increased adenovirus-associated but not cytomegalovirus-associated mortality in children with severe acute graft-versus-host disease. Stem Cells Transl Med, 2014. 3(8): p. 899-910.
44. von Bahr, V., et al., Mesenchymal stem cells may ameliorate inflammation in an ex vivo model of extracorporeal membrane oxygenation. Perfusion, 2019. 34(1_suppl): p. 15-21.
45. Huppert, L. A., K. D. Liu, and M. A. Matthay, Therapeutic potential of mesenchymal stromal cells in the treatment of ARDS. Transfusion, 2019. 59(S1): p. 869-875.
46. Jung, Y. J., et al., The effect of human adipose-derived stem cells on lipopolysaccharide-induced acute respiratory distress syndrome in mice. Ann Transl Med, 2019. 7(22): p. 674.
47. Chen, C. H., et al., Effective protection against acute respiratory distress syndrome/sepsis injury by combined adipose-derived mesenchymal stem cells and preactivated disaggregated platelets. Oncotarget, 2017. 8(47): p. 82415-82429.
48. Lu, H., et al., Pulmonary Retention of Adipose Stromal Cells Following Intravenous Delivery Is Markedly Altered in the Presence of ARDS. Cell Transplant, 2016. 25(9): p. 1635-1643.
49. Zheng, G., et al., Treatment of acute respiratory distress syndrome with allogeneic adipose-derived mesenchymal stem cells: a randomized, placebo-controlled pilot study. Respir Res, 2014. 15: p. 39.
50. Lu, Z., et al., Mesenchymal stem cells induce dendritic cell immune tolerance via paracrine hepatocyte growth factor to alleviate acute lung injury. Stem Cell Res Ther, 2019. 10(1): p. 372.
51. Silva, J. D., et al., Mesenchymal Stromal Cells Are More Effective Than Their Extracellular Vesicles at Reducing Lung Injury Regardless of Acute Respiratory Distress Syndrome Etiology. Stem Cells Int, 2019. 2019: p. 8262849.
52. Xu, A. L., et al., Mesenchymal Stem Cells Reconditioned in Their Own Serum Exhibit Augmented Therapeutic Properties in the Setting of Acute Respiratory Distress Syndrome. Stem Cells Transl Med, 2019. 8(10): p. 1092-1106.
53. Cardenes, N., et al., Cell therapy for ARDS: efficacy of endobronchial versus intravenous administration and biodistribution of MAPCs in a large animal model. BMJ Open Respir Res, 2019. 6(1): p. e000308.
54. Li, L., et al., Mesenchymal stem cells with downregulated Hippo signaling attenuate lung injury in mice with lipopolysaccharideinduced acute respiratory distress syndrome. Int J Mol Med, 2019. 43(3): p. 1241-1252.
55. Mokhber Dezfouli, M. R., et al., Intrapulmonary autologous transplant of bone marrow-derived mesenchymal stromal cells improves lipopolysaccharide-induced acute respiratory distress syndrome in rabbit. Crit Care, 2018. 22(1): p. 353.
56. Schwede, M., et al., Effects of bone marrow-derived mesenchymal stromal cells on gene expression in human alveolar type II cells exposed to TNF-alpha, IL-1 beta, and IFN-gamma. Physiol Rep, 2018. 6(16): p. e13831.
57. Masterson, C., et al., Syndecan-2-positive, Bone Marrow-derived Human Mesenchymal Stromal Cells Attenuate Bacterial-induced Acute Lung Injury and Enhance Resolution of Ventilator-induced Lung Injury in Rats. Anesthesiology, 2018. 129(3): p. 502-516.
58. Park, J., et al., Expression profile of microRNAs following bone marrow-derived mesenchymal stem cell treatment in lipopolysaccharide-induced acute lung injury. Exp Ther Med, 2018. 15(6): p. 5495-5502.
59. Pedrazza, L., et al., Mesenchymal stem cells improves survival in LPS-induced acute lung injury acting through inhibition of NETs formation. J Cell Physiol, 2017. 232(12): p. 3552-3564.
60. Yang, Y., et al., The Vascular Endothelial Growth Factors-Expressing Character of Mesenchymal Stem Cells Plays a Positive Role in Treatment of Acute Lung Injury In vivo. Mediators Inflamm, 2016. 2016: p. 2347938.
61. Moodley, Y., et al., Human mesenchymal stem cells attenuate early damage in a ventilated pig model of acute lung injury. Stem Cell Res, 2016. 17(1): p. 25-31.
62. Hayes, M., et al., Mesenchymal stromal cells are more effective than the MSC secretome in diminishing injury and enhancing recovery following ventilator-induced lung injury. Intensive Care Med Exp, 2015. 3(1): p. 29.
63. Monsel, A., et al., Therapeutic Effects of Human Mesenchymal Stem Cell-derived Microvesicles in Severe Pneumonia in Mice. Am J Respir Crit Care Med, 2015. 192(3): p. 324-36.
64. Hao, Q., et al., Study of Bone Marrow and Embryonic Stem Cell-Derived Human Mesenchymal Stem Cells for Treatment of Escherichia coli Endotoxin-Induced Acute Lung Injury in Mice. Stem Cells Transl Med, 2015. 4(7): p. 832-40.
65. Devaney, J., et al., Human mesenchymal stromal cells decrease the severity of acute lung injury induced by E. coli in the rat. Thorax, 2015. 70(7): p. 625-35.
66. Asmussen, S., et al., Human mesenchymal stem cells reduce the severity of acute lung injury in a sheep model of bacterial pneumonia. Thorax, 2014. 69(9): p. 819-25.
67. Shalaby, S. M., et al., Mesenchymal stromal cell injection protects against oxidative stress in Escherichia coli-induced acute lung injury in mice. Cytotherapy, 2014. 16(6): p. 764-75.
68. Bustos, M. L., et al., Activation of human mesenchymal stem cells impacts their therapeutic abilities in lung injury by increasing interleukin (IL)-10 and IL-1RN levels. Stem Cells Transl Med, 2013. 2(11): p. 884-95.
69. Rojas, M., et al., Infusion of freshly isolated autologous bone marrow derived mononuclear cells prevents endotoxin-induced lung injury in an ex-vivo perfused swine model. Stem Cell Res Ther, 2013. 4(2): p. 26.
70. Yan, X., et al., Nrf2/Keap1/ARE Signaling Mediated an Antioxidative Protection of Human Placental Mesenchymal Stem Cells of Fetal Origin in Alveolar Epithelial Cells. Oxid Med Cell Longev, 2019. 2019: p. 2654910.
71. Cui, P., et al., Human amnion-derived mesenchymal stem cells alleviate lung injury induced by white smoke inhalation in rats. Stem Cell Res Ther, 2018. 9(1): p. 101.
72. Zhang, S., et al., Nrf2 transfection enhances the efficacy of human amniotic mesenchymal stem cells to repair lung injury induced by lipopolysaccharide. J Cell Biochem, 2018. 119(2): p. 1627-1636.
73. Huang, Z., et al., Transcriptomic analysis of lung tissues after hUC-MSCs and FTY720 treatment of lipopolysaccharide-induced acute lung injury in mouse models. Int Immunopharmacol, 2018. 63: p. 26-34.
74. Xuan, Y. Y., et al., Human Mesenchymal Stem/Stromal Cells From Human Umbilical Cord Ameliorate Acute Respiratory Distress Syndrome in Rats: Factors to Consider. Crit Care Med, 2017. 45(7): p. e736-e737.
75. Lee, F. Y., et al., Xenogeneic human umbilical cord-derived mesenchymal stem cells reduce mortality in rats with acute respiratory distress syndrome complicated by sepsis. Oncotarget, 2017. 8(28): p. 45626-45642.
76. Zhu, H., et al., Therapeutic Effects of Human Umbilical Cord-Derived Mesenchymal Stem Cells in Acute Lung Injury Mice. Sci Rep, 2017. 7: p. 39889.
77. Curley, G. F., et al., Cryopreserved, Xeno-Free Human Umbilical Cord Mesenchymal Stromal Cells Reduce Lung Injury Severity and Bacterial Burden in Rodent Escherichia coli-Induced Acute Respiratory Distress Syndrome. Crit Care Med, 2017. 45(2): p. e202-e212.
78. Chang, Y., et al., Intratracheal administration of umbilical cord blood-derived mesenchymal stem cells in a patient with acute respiratory distress syndrome. J Korean Med Sci, 2014. 29(3): p. 438-40.
79. Moodley, Y., et al., Human umbilical cord mesenchymal stem cells reduce fibrosis of bleomycin-induced lung injury. Am J Pathol, 2009. 175(1): p. 303-13.
80. Xiang, B., et al., Transplantation of Menstrual Blood-Derived Mesenchymal Stem Cells Promotes the Repair of LPS-Induced Acute Lung Injury. Int J Mol Sci, 2017. 18(4).
81. Wang, L., et al., Lung-Resident Mesenchymal Stem Cells Promote Repair of LPS-Induced Acute Lung Injury via Regulating the Balance of Regulatory T cells and Th17 cells. Inflammation, 2019. 42(1): p. 199-210.
82. Silva, J. D., et al., Mesenchymal Stem Cells From Bone Marrow, Adipose Tissue, and Lung Tissue Differentially Mitigate Lung and Distal Organ Damage in Experimental Acute Respiratory Distress Syndrome. Crit Care Med, 2018. 46(2): p. e132-e140.
83. Su, V. Y., et al., Mesenchymal Stem Cell-Conditioned Medium Induces Neutrophil Apoptosis Associated with Inhibition of the NF-kappaB Pathway in Endotoxin-Induced Acute Lung Injury. Int J Mol Sci, 2019. 20(9).
84. Mohammadipoor, A., et al., Therapeutic potential of products derived from mesenchymal stem/stromal cells in pulmonary disease. Respir Res, 2018. 19(1): p. 218.
85. Lee, J. H., J. Park, and J. W. Lee, Therapeutic use of mesenchymal stem cell-derived extracellular vesicles in acute lung injury. Transfusion, 2019. 59(S1): p. 876-883.
86. Abreu, S. C., D. J. Weiss, and P. R. Rocco, Extracellular vesicles derived from mesenchymal stromal cells: a therapeutic option in respiratory diseases? Stem Cell Res Ther, 2016. 7(1): p. 53.
87. Monsel, A., et al., Mesenchymal stem cell derived secretome and extracellular vesicles for acute lung injury and other inflammatory lung diseases. Expert Opin Biol Ther, 2016. 16(7): p. 859-71.
88. Liu, F. B., Q. Lin, and Z. W. Liu, A study on the role of apoptotic human umbilical cord mesenchymal stem cells in bleomycin-induced acute lung injury in rat models. Eur Rev Med Pharmacol Sci, 2016. 20(5): p. 969-82.
89. Chen, J., et al., Mesenchymal Stem Cell Conditioned Medium Promotes Proliferation and Migration of Alveolar Epithelial Cells under Septic Conditions In vitro via the JNK-P38 Signaling Pathway. Cell Physiol Biochem, 2015. 37(5): p. 1830-46.
90. Ionescu, L., et al., Stem cell conditioned medium improves acute lung injury in mice: in vivo evidence for stem cell paracrine action. Am J Physiol Lung Cell Mol Physiol, 2012. 303(11): p. L967-77.
91. Wang, D., et al., Clinical Characteristics of 138 Hospitalized Patients With 2019 Novel Coronavirus-Infected Pneumonia in Wuhan, China. JAMA, 2020.
92. Channappanavar, R. and S. Perlman, Pathogenic human coronavirus infections: causes and consequences of cytokine storm and immunopathology. Semin Immunopathol, 2017. 39(5): p. 529-539.
93. Isaacs, A. and J. Lindenmann, Virus interference. I. The interferon. Proc R Soc Lond B Biol Sci, 1957. 147(927): p. 258-67.
94. Isaacs, A., J. Lindenmann, and R. C. Valentine, Virus interference. II. Some properties of interferon. Proc R Soc Lond B Biol Sci, 1957. 147(927): p. 268-73.
95. Johnson, H. M., Differentiation of the immunosuppressive and antiviral effects of interferon. Cell Immunol, 1978. 36(2): p. 220-30.
96. Minagawa, H., et al., Protective effect of recombinant murine interferon beta against mouse hepatitis virus infection. Antiviral Res, 1987. 8(2): p. 85-95.
97. van Seventer, J. M., T. Nagai, and G. A. van Seventer, Interferon-beta differentially regulates expression of the IL-12 family members p35, p40, p19 and EBI3 in activated human dendritic cells. J Neuroimmunol, 2002. 133(1-2): p. 60-71.
98. Hermanrud, C., et al., Different Interferon Beta Preparations Induce the Same Qualitative Immune Response in Human Skin. J Interferon Cytokine Res, 2019. 39(5): p. 302-313.
99. Kadowaki, N., et al., Natural interferon alpha/beta-producing cells link innate and adaptive immunity. J Exp Med, 2000. 192(2): p. 219-26.
100. Cremer, I., et al., Inhibition of human immunodeficiency virus transmission to CD4+ T cells after gene transfer of constitutively expressed interferon beta to dendritic cells. Hum Gene Ther, 2000. 11(12): p. 1695-703.
101. Izuma, M., et al., In vitro cytokine production of peripheral blood mononuclear cells in response to HCV core antigen stimulation during interferon-beta treatment and its relevance to sCD8 and sCD30. Hepatol Res, 2000. 18(3): p. 218-229.
102. Dalod, M., et al., Interferon alpha/beta and interleukin 12 responses to viral infections: pathways regulating dendritic cell cytokine expression in vivo. J Exp Med, 2002. 195(4): p. 517-28.
103. Honda, K., et al., Selective contribution of IFN-alpha/beta signaling to the maturation of dendritic cells induced by double-stranded RNA or viral infection. Proc Natl Acad Sci USA, 2003. 100(19): p. 10872-7.
104. Byrnes, A. A., et al., Type I interferons and IL-12: convergence and cross-regulation among mediators of cellular immunity. Eur J Immunol, 2001. 31(7): p. 2026-34.
105. McNally, J. M., et al., Attrition of bystander CD8 T cells during virus-induced T-cell and interferon responses. J Virol, 2001. 75(13): p. 5965-76.
106. Biron, C. A., Role of early cytokines, including alpha and beta interferons (IFN-alpha/beta), in innate and adaptive immune responses to viral infections. Semin Immunol, 1998. 10(5): p. 383-90.
107. Sareneva, T., et al., Influenza A virus-induced IFN-alpha/beta and IL-18 synergistically enhance IFN-gamma gene expression in human T cells. J Immunol, 1998. 160(12): p. 6032-8.
108. Brown, J. L., J. Barsoum, and X. Q. Qin, CD4+ T helper cell-independent antitumor response mediated by murine IFN-beta gene delivery in immunocompetent mice. J Interferon Cytokine Res, 2002. 22(6): p. 719-28.
109. Johnson, H. M. and S. Baron, Interferon: effects on the immune response and the mechanism of activation of the cellular response. CRC Crit Rev Biochem, 1976. 4(2): p. 203-27.
110. Kuroki, M., et al., Antitumor effect of recombinant murine interferon-beta against mouse malignant glioma. J Interferon Res, 1987. 7(3): p. 301-11.
111. Markovic, S. N., P. R. Knight, and D. M. Murasko, Inhibition of interferon stimulation of natural killer cell activity in mice anesthetized with halothane or isoflurane. Anesthesiology, 1993. 78(4): p. 700-6.
112. Zdravkovic, M., et al., In vitro cytotoxic activity of cord blood NK cells against herpes simplex virus type-1 infected purified human term villous cytotrophoblast. Viral Immunol, 1994. 7(3): p. 133-40.
113. Biron, C. A., Natural killer cell regulation during viral infection. Biochem Soc Trans, 1997. 25(2): p. 687-90.
114. Weinberg, A., L. Zhang, and R. M. Tuder, NK recognition of cytomegalovirus-infected endothelial cells depends on viral replication and MHC class I expression. Viral Immunol, 1996. 9(2): p. 131-40.
115. Sato, K., et al., Antiviral response by natural killer cells through TRAIL gene induction by IFN-alpha/beta. Eur J Immunol, 2001. 31(11): p. 3138-46.
116. Nguyen, K. B., et al., Coordinated and distinct roles for IFN-alpha beta, IL-12, and IL-15 regulation of NK cell responses to viral infection. J Immunol, 2002. 169(8): p. 4279-87.
117. Wang, B. X. and E. N. Fish, Global virus outbreaks: Interferons as 1st responders. Semin Immunol, 2019. 43: p. 101300.
118. Konde, M. K., et al., Interferon beta-1a for the treatment of Ebola virus disease: A historically controlled, single-arm proof-of-concept trial. PLoS One, 2017. 12(2): p. e0169255.
119. Wrensch, F., et al., Interferon-Induced Transmembrane Protein-Mediated Inhibition of Host Cell Entry of Ebolaviruses. J Infect Dis, 2015. 212 Suppl 2: p. S210-8.
120. Shuchman, M., Could interferon help treat Ebola? CMAJ, 2014. 186(16): p. 1204.
121. Smith, L. M., et al., Interferon-beta therapy prolongs survival in rhesus macaque models of Ebola and Marburg hemorrhagic fever. J Infect Dis, 2013. 208(2): p. 310-8.
122. Al-Tawfiq, J. A., et al., Ribavirin and interferon therapy in patients infected with the Middle East respiratory syndrome coronavirus: an observational study. Int J Infect Dis, 2014. 20: p. 42-6.
123. Faure, E., et al., Distinct immune response in two MERS-CoV-infected patients: can we go from bench to bedside? PLoS One, 2014. 9(2): p. e88716.
124. Khalid, M., et al., Ribavirin and interferon-alpha2b as primary and preventive treatment for Middle East respiratory syndrome coronavirus: a preliminary report of two cases. Antivir Ther, 2015. 20(1): p. 87-91.
125. Kim, U. J., et al., Combination therapy with lopinavir/ritonavir, ribavirin and interferon-alpha for Middle East respiratory syndrome. Antivir Ther, 2016. 21(5): p. 455-9.
126. Mayer, K., et al., Fatal outcome of human coronavirus NL63 infection despite successful viral elimination by IFN-alpha in a patient with newly diagnosed ALL. Eur J Haematol, 2016. 97(2): p. 208-210.

What is claimed is:

1. A method of treating or preventing acute respiratory distress syndrome (ARDS) in a subject comprising administering to the subject an effective amount of fibroblasts and/or fibroblast-derived products.

2. The method of claim 1, wherein the method comprises providing to the subject an effective amount of fibroblasts.

3. The method of claim 1, wherein the method comprises providing to the subject an effective amount of fibroblast-derived products.

4. The method of claim 3, wherein the fibroblast-derived products comprise conditioned media derived from fibroblasts.

5. The method of claim 3, wherein the fibroblast-derived products comprise microvesicles from fibroblasts.

6. The method of claim 3, wherein the fibroblast-derived products comprise exosomes from fibroblasts.

7. The method of claim 3, wherein the fibroblast-derived products comprise apoptotic vesicles from fibroblasts.

8. The method of claim 3, wherein the fibroblast-derived products comprise nucleic acids from fibroblasts.

9. The method of any one of claims 1-8, wherein the fibroblasts are derived from a source of tissue selected from the group consisting of dermal tissue; placental tissue; hair follicle; deciduous tooth; omentum; placenta; Wharton's jelly; bone marrow; adipose tissue; amniotic membrane; amniotic fluid; peripheral blood; and a combination thereof.

10. The method of claim 9, wherein the peripheral blood comprises mobilized peripheral blood.

11. The method of claim 10, wherein the mobilized peripheral blood comprises peripheral blood from an individual that is treated with G-CSF; M-CSF; GM-CSF; Mozibil; flt-3 ligand; or a combination thereof.

12. The method of any one of claims 1-11, wherein the fibroblasts, with respect to the subject, are allogeneic; autologous; xenogeneic; or a combination thereof.

13. The method of any one of claims 1-12, wherein the ARDS is caused by one or more factors selected from the group consisting of cytokine storm; immunological cell infiltration; bacterial infection; viral infection; systemic inflammatory response syndrome; systemic inflammation; acute radiation syndrome; sepsis; and a combination thereof.

14. The method of any one of claims 1-13, wherein the fibroblasts are administered intravenously.

15. The method of any one of claims 1-14, wherein the fibroblasts are administered intranasally.

16. The method of any one of claims 1-15, wherein the fibroblasts are administered intratracheally.

17. The method of any one of claims 1-16, wherein the fibroblasts are pre-activated with one or more agents capable of enhancing a fibroblast therapeutic activity.

18. The method of claim 17, wherein the fibroblast therapeutic activity comprises a mobility towards a chemotactic agent; a production of anti-inflammatory molecules; a production of anti-apoptotic molecules; or a combination thereof.

19. The method of claim 18, wherein the mobility towards a chemotactic agent is mediated by enhanced expression of one or more receptors associated with enhanced chemotaxis.

20. The method of claim 19, wherein the receptor associated with enhanced chemotaxis comprises CXCR4.

21. The method of claim 18, wherein the production of anti-inflammatory molecules comprises the production of molecules selected from the group consisting of IL-4; IL-10; IL-13; IL-20; IL-27; IL-35; PGE-2; indolamine 2,3 deoxygenase; TGF-beta; EGF; and a combination thereof.

22. The method of any one of claims 1-21, wherein the fibroblasts are modified to express enhanced levels of one or more therapeutic cytokines.

23. The method of claim 22, wherein the one or more therapeutic cytokines are selected from the group consisting of cytokines that inhibit apoptosis; cytokines that act as growth factors; cytokines that act as immune modulators and/or anti-inflammatory agents; and a combination thereof.

24. The method of claim 23, wherein the cytokines that inhibit apoptosis are selected from the group consisting of EGF; VEGF; angiopoietin; and a combination thereof.

25. The method of claim 23, wherein the cytokines that act as growth factors are selected from the group consisting of HGF; FGF-1; FGF-2; KGF; CTNF; and a combination thereof.

26. The method of claim 23, wherein the cytokines that act as immune modulators and/or anti-inflammatory agents are selected from the group consisting of IL-4; IL-10; IL-13; IL-20; IL-27; IL-35; PGE-2; indolamine 2,3 deoxygenase; TGF-beta; neuroaminidase; and a combination thereof.

27. The method of any one of claims 1-26, further comprising administering a second treatment to the subject, wherein the second treatment is administered to the subject sequentially and/or simultaneously with the effective amount of fibroblasts and/or fibroblast-derived products.

28. The method of claim 27, wherein the second treatment comprises ventilation, a glucocorticoid, a surfactant, inhaled nitric oxide, an antioxidant, a protease inhibitor, a recombinant human activated protein C, a β2-agonist, lisofylline, a statin, inhaled heparin, a diuretic, a sedative, an analgesic, a muscle relaxant, an antibiotic, inhaled prostacyclin, inhaled synthetic prostacydin analog, ketoconazole, alprostadil, keratinocyte growth factor, beta-agonists, human monoclonal antibody (mAb) against tissue factor VIIa (TS factor 7a), interferon receptor agonists, insulin, perfluorocarbon, budesonide, recombinant human angiotensin-converting enzyme (ACE), recombinant human Clara cell 10 kDa (CC10) protein, tissue plasminogen activator, human mesenchymal stem cells, nutritional therapy, or a combination thereof.

29. The method of claim 27, wherein the second treatment comprises methylprednisolone, dexamethasone, prednisone, prednisolone, betamethasone, triamcinolone, triamcinolone acetonide, beclometasone, albuterol, lisofylline, rosuvastatin, inhaled heparin, inhaled nitric oxide, recombinant human activated protein C, ibuprofen, naproxen, acetaminophen, cisatracurium besylate, procysteine, acetylcysteine, inhaled prostacydin, ketoconazole, alprostadil, keratinocyte growth factor, beta-agonists, human monoclonal antibody (mAb) against tissue factor VIIa (TS factor 7a), insulin, perfluorocarbon, budesonide, recombinant human angiotensin-converting enzyme (ACE), recombinant human Clara cell 10 kDa (CC10) protein, tissue plasminogen activator, human mesenchymal stem cells, a nutritional therapy, a combination of omega-3 fatty acids, antioxidants, gamma-linolenic acids with isocaloric foods, mechanical ventilation, or a combination thereof.

30. The methods of any one of claims 1-29, wherein the fibroblasts comprise viral infection suppression activity.

31. The method of claim 30, wherein the fibroblasts produce interferon.

32. The method of claim 31, wherein the interferon comprises interferon alpha; interferon beta; interferon gamma; interferon tau; interferon omega; or a combination thereof.

33. The method of claim 30, wherein the fibroblasts suppress viral infection upon contact with one or more activators of a toll like receptor.

34. The method of claim 33, wherein the toll like receptor comprises TLR-1.

35. The method of claim 34, wherein the activator of TLR-1 comprises Pam3CSK4.

36. The method of claim 33, wherein the toll like receptor comprises TLR-2.

37. The method of claim 36, wherein the activator of TLR-2 comprises HKLM.

38. The method of claim 33, wherein the toll like receptor comprises TLR-3.

39. The method of claim 38, wherein the activator of TLR-3 comprises Poly:IC.

40. The method of claim 33, wherein the toll like receptor comprises TLR-4.

41. The method of claim 40, wherein the activator of TLR-4 comprises LPS.

42. The method of claim 40, wherein the activator of TLR-4 comprises buprenorphine.

43. The method of claim 40, wherein the activator of TLR-4 comprises carbamazepine.

44. The method of claim 40, wherein the activator of TLR-4 comprises fentanyl.

45. The method of claim 40, wherein the activator of TLR-4 comprises levorphanol.

46. The method of claim 40, wherein the activator of TLR-4 comprises methadone.

47. The method of claim 40, wherein the activator of TLR-4 comprises cocaine.

48. The method of claim 40, wherein the activator of TLR-4 comprises morphine.

49. The method of claim 40, wherein the activator of TLR-4 comprises oxcarbazepine.

50. The method of claim 40, wherein the activator of TLR-4 comprises oxycodone.

51. The method of claim 40, wherein the activator of TLR-4 comprises pethidine.

52. The method of claim 40, wherein the activator of TLR-4 comprises glucuronoxylomannan from Cryptococcus.

53. The method of claim 40, wherein the activator of TLR-4 comprises morphine-3-glucuronide.

54. The method of claim 40, wherein the activator of TLR-4 comprises lipoteichoic acid.

55. The method of claim 40, wherein the activator of TLR-4 comprises β-defensin 2.

56. The method of claim 40, wherein the activator of TLR-4 comprises small molecular weight hyaluronic acid.

57. The method of claim 40, wherein the activator of TLR-4 comprises fibronectin EDA.

58. The method of claim 40, wherein the activator of TLR-4 comprises snapin.

59. The method of claim 40, wherein the activator of TLR-4 comprises tenascin C.

60. The method of claim 33, wherein the toll like receptor comprises TLR-5.

61. The method of claim 60, wherein the activator of TLR-5 comprises flagellin.

62. The method of claim 33, wherein the toll like receptor comprises TLR-6.

63. The method of claim 62, wherein the activator of TLR-6 comprises FSL-1.

64. The method of claim 33, wherein the toll like receptor comprises TLR-7.

65. The method of claim 64, wherein the activator of TLR-7 comprises imiquimod.

66. The method of claim 33, wherein the toll like receptor of TLR-8.

67. The method of claim 66, wherein the activator of TLR8 comprises ssRNA40/LyoVec.

68. The method of claim 33, wherein the toll like receptor of TLR-9.

69. The method of claim 68, wherein the activator of TLR-9 comprises a CpG oligonucleotide.

70. The method of claim 68, wherein the activator of TLR-9 comprises ODN2006.

71. The method of claim 68, wherein the activator of TLR-9 comprises agatolimod.

72. A method of reprogramming monocytes in the lung of a subject having, or at risk of having, ARDS, comprising administering to the subject an effective amount of fibroblasts and/or fibroblast-derived products.

73. The method of claim 72, wherein the method comprises providing to the subject an effective amount of fibroblasts.

74. The method of claim 72, wherein the method comprises providing to the subject an effective amount of fibroblast-derived products.

75. The method of claim 74, wherein the fibroblast-derived products comprise conditioned media derived from fibroblasts.

76. The method of claim 74, wherein the fibroblast-derived products comprise microvesicles from fibroblasts.

77. The method of claim 74, wherein the fibroblast-derived products comprise exosomes from fibroblasts.

78. The method of claim 74, wherein the fibroblast-derived products comprise apoptotic vesicles from fibroblasts.

79. The method of claim 74, wherein the fibroblast-derived products comprise nucleic acids from fibroblasts.

80. The method of any one of claims 72-79, wherein the fibroblasts are derived from a source of tissue selected from the group consisting of dermal; placental; hair follicle; deciduous tooth; omentum; placenta; Wharton's jelly; bone marrow; adipose tissue; amniotic membrane; amniotic fluid; peripheral blood; and a combination thereof.

81. The method of claim 80, wherein the peripheral blood comprises mobilized peripheral blood.

82. The method of claim 81, wherein the mobilized peripheral blood comprises peripheral blood from an individual that is treated with G-CSF; M-CSF; GM-CSF; Mozibil; flt-3 ligand; or a combination thereof.

83. The method of any one of claims 72-82, wherein the fibroblasts, with respect to the subject, are allogeneic; autologous; xenogeneic; or a combination thereof.

84. The method of any one of claims 72-83, wherein the ARDS is caused by one or more factors selected from the group consisting of cytokine storm; immunological cell infiltration; bacterial infection; viral infection; systemic inflammatory response syndrome; systemic inflammation; acute radiation syndrome; sepsis; and a combination thereof.

85. The method of any one of claims 72-84, wherein the fibroblasts are administered to the subject intravenously.

86. The method of any one of claims 72-85, wherein the fibroblasts are administered to the subject intranasally.

87. The method of any one of claims 72-86, wherein the fibroblasts are administered to the subject intratracheally.

88. The method of any one of claims 72-87, wherein the fibroblasts are pre-activated with one or more agents capable of enhancing a fibroblast therapeutic activity.

89. The method of claim 88, wherein the fibroblast therapeutic activity comprises a mobility towards a chemotactic agent; a production of anti-inflammatory molecules; a production of anti-apoptotic molecules; or a combination thereof.

90. The method of claim 89, wherein the mobility towards a chemotactic agent is mediated by enhanced expression of one or more receptors associated with enhanced chemotaxis.

91. The method of claim 90, wherein the receptor associated with enhanced chemotaxis comprises CXCR4.

92. The method of claim 89, wherein the production of anti-inflammatory molecules comprises the production of molecules selected from the group consisting of IL-4; IL-10; IL-13; IL-20; IL-27; IL-35; PGE-2; indolamine 2,3 deoxygenase; TGF-beta; EGF; and a combination thereof.

93. The method of any one of claims 72-92, wherein the fibroblasts are modified to express enhanced levels of one or more therapeutic cytokines.

94. The method of claim 93, wherein the one or more therapeutic cytokines are selected from the group consisting of cytokines that inhibit apoptosis; cytokines that act as growth factors; cytokines that act as immune modulators and/or anti-inflammatory agents; and a combination thereof.

95. The method of claim 94, wherein the cytokines that inhibit apoptosis are selected from the group consisting of EGF; VEGF; angiopoietin; and a combination thereof.

96. The method of claim 94, wherein the cytokines that act as growth factors are selected from the group consisting of HGF; FGF-1; FGF-2; KGF; CTNF; and a combination thereof.

97. The method of claim 94, wherein the cytokines that act as immune modulators and/or anti-inflammatory agents are selected from the group consisting of: IL-4; IL-10; IL-13; IL-20; IL-27; IL-35; PGE-2; indolamine 2,3 deoxygenase; TGF-beta; neuroaminidase; and a combination thereof.

98. The method of any one of claims 72-97, further comprising administering a second treatment to the subject, wherein the second treatment is administered to the subject sequentially and/or simultaneously with the effective amount of fibroblasts and/or fibroblast-derived products.

99. The method of claim 98, wherein the second treatment comprises ventilation, a glucocorticoid, a surfactant, inhaled nitric oxide, an antioxidant, a protease inhibitor, a recombinant human activated protein C, a β2-agonist, lisofylline, a statin, inhaled heparin, a diuretic, a sedative, an analgesic, a muscle relaxant, an antibiotic, inhaled prostacyclin, inhaled synthetic prostacydin analog, ketoconazole, alprostadil, keratinocyte growth factor, beta-agonists, human monoclonal antibody (mAb) against tissue factor VIIa (TS factor 7a), interferon receptor agonists, insulin, perfluorocarbon, budesonide, recombinant human angiotensin-converting enzyme (ACE), recombinant human Clara cell 10 kDa (CC10) protein, tissue plasminogen activator, human mesenchymal stem cells, nutritional therapy, or a combination thereof.

100. The method of claim 98, wherein the second treatment comprises methylprednisolone, dexamethasone, prednisone, prednisolone, betamethasone, triamcinolone, triamcinolone acetonide, beclometasone, albuterol, lisofylline, rosuvastatin, inhaled heparin, inhaled nitric oxide, recombinant human activated protein C, ibuprofen, naproxen, acetaminophen, cisatracurium besylate, procysteine, acetylcysteine, inhaled prostacydin, ketoconazole, alprostadil, keratinocyte growth factor, beta-agonists, human monoclonal antibody (mAb) against tissue factor VIIa (TS factor 7a), insulin, perfluorocarbon, budesonide, recombinant human angiotensin-converting enzyme (ACE), recombinant human Clara cell 10 kDa (CC10) protein, tissue plasminogen activator, human mesenchymal stem cells, a nutritional therapy, a combination of omega-3 fatty acids, antioxidants, gamma-linolenic acids with isocaloric foods, mechanical ventilation, or a combination thereof.

101. The methods of any one of claims 72-100, wherein the fibroblasts comprise viral infection suppression activity.

102. The method of claim 101, wherein the fibroblasts produce interferon.

103. The method of claim 102, wherein the interferon comprises interferon alpha; interferon beta; interferon gamma; interferon tau; interferon omega; or a combination thereof.

104. The method of claim 101, wherein the fibroblasts suppress viral infection upon contact with one or more activators of a toll like receptor.

105. The method of claim 104, wherein the toll like receptor comprises TLR-1.

106. The method of claim 105, wherein the activator of TLR-1 comprises Pam3CSK4.

107. The method of claim 104, wherein the toll like receptor comprises TLR-2.

108. The method of claim 107, wherein the activator of TLR-2 comprises HKLM.

109. The method of claim 104, wherein the toll like receptor comprises TLR-3.

110. The method of claim 109, wherein the activator of TLR-3 comprises Poly:IC.

111. The method of claim 104, wherein the toll like receptor comprises TLR-4.

112. The method of claim 111, wherein the activator of TLR-4 comprises LPS.

113. The method of claim 111, wherein the activator of TLR-4 comprises buprenorphine.

114. The method of claim 111, wherein the activator of TLR-4 comprises carbamazepine.

115. The method of claim 111, wherein the activator of TLR-4 comprises fentanyl.

116. The method of claim 111, wherein the activator of TLR-4 comprises levorphanol.

117. The method of claim 111, wherein the activator of TLR-4 comprises methadone.

118. The method of claim 111, wherein the activator of TLR-4 comprises cocaine.

119. The method of claim 111, wherein the activator of TLR-4 comprises morphine.

120. The method of claim 111, wherein the activator of TLR-4 comprises oxcarbazepine.

121. The method of claim 111, wherein the activator of TLR-4 comprises oxycodone.

122. The method of claim 111, wherein the activator of TLR-4 comprises pethidine.

123. The method of claim 111, wherein the activator of TLR-4 comprises glucuronoxylomannan from Cryptococcus.

124. The method of claim 111, wherein the activator of TLR-4 comprises morphine-3-glucuronide.

125. The method of claim 111, wherein the activator of TLR-4 comprises lipoteichoic acid.

126. The method of claim 111, wherein the activator of TLR-4 comprises (3-defensin 2.

127. The method of claim 111, wherein the activator of TLR-4 comprises small molecular weight hyaluronic acid.

128. The method of claim 111, wherein the activator of TLR-4 comprises fibronectin EDA.

129. The method of claim 111, wherein the activator of TLR-4 comprises snapin.

130. The method of claim 111, wherein the activator of TLR-4 comprises tenascin C.

131. The method of claim 104, wherein the toll like receptor comprises TLR-5.

132. The method of claim 131, wherein the activator of TLR-5 comprises flagellin.

133. The method of claim 104, wherein the toll like receptor comprises TLR-6.

134. The method of claim 133, wherein the activator of TLR-6 comprises FSL-1.

135. The method of claim 104, wherein the toll like receptor comprises TLR-7.

136. The method of claim 135, wherein the activator of TLR-7 comprises imiquimod.

137. The method of claim 104, wherein the toll like receptor of TLR-8.

138. The method of claim 137, wherein the activator of TLR8 comprises ssRNA40/LyoVec.

139. The method of claim 104, wherein the toll like receptor of TLR-9.

140. The method of claim 139, wherein the activator of TLR-9 comprises a CpG oligonucleotide.

141. The method of claim 139, wherein the activator of TLR-9 comprises ODN2006.

142. The method of claim 139, wherein the activator of TLR-9 comprises agatolimod.
